(19) United States
(12) Patent Application Publication
(10) Pub. No.: US 2003/0229137 A1
(43) Pub. Date: Dec. 11, 2003

(54) Methods of inhibiting osteoclast activity

(76) Inventors: Shoujun Chen, Bedford, MA (US); Joel McCleary, The Plains, VA (US); Lijun Sun, Harvard, MA (US)

Correspondence Address: FISH & RICHARDSON PC, 225 FRANKLIN ST, BOSTON, MA, 02110, US

(21) Appl. No.: 10/269,722

(22) Filed: Oct. 11, 2002

Related U.S. Application Data
(60) Provisional application No. 60/328,986, filed on Oct. 12, 2001.

(51) Int. Cl. A61K 31/366

(52) U.S. Cl. 514/456; 514/460

(57) ABSTRACT

Kavalactones having osteoclast inhibitory activity are described. The kavalactones, and compositions thereof, are useful in mediating osteoclast activity, and as such, are useful in treating or preventing disease or disease symptoms mediated by osteoclast activity.

### CROSS-REFERENCE TO RELATED APPLICATIONS

This application claims benefit of U.S. application serial No. 60/328,986, filed, Oct. 12, 2001, which is herein incorporated by reference in its entirety.

### BACKGROUND

It is believed that the use of kava (Piper methysticum Forst.) predates written history. The origination of the plant is attributed to the New Guinea/Indonesia area and it is believed that Polynesian explorers were responsible for its spread from island to island. Oceania (i.e., the Pacific island communities of Micronesia, Melanesia and Polynesia) is an area where islanders have been known for centuries to consume a drink, also called kava and derived from the root of kava, in ceremonies and celebrations due to its reported calming effect and ability to promote sociability. The root and the drink were apparently first described in the Western world by Captain James Cook as a result of his exploration of the South Seas in 1768. Many myths and anecdotal stories surround the use of kava, and these vary from culture to culture.

The extract of the kava root is known to contain a class of structurally related chemical compounds known as kavalactones. At least sixteen different members of this chemical class are known to be present. A relaxing action (i.e., calming effect, sleep inducing effect) of the extract is attributed to certain members of this class. Certain inflammatory responses are also attributed to kava extract. The isolated kavalactone (+)-kawain inhibited arachidonic-acid (AA)-stimulated aggregation in human platelets, and also inhibited adenosine triphosphate (ATP)-induced exocytosis, cyclooxygenase activity and thromboxane synthetase activity. In platelet preparations, application of (+)-kawain five minutes before application of AA diminished aggregation, ATP release, and synthesis of TXA₂ and PGE₂ (IC50 values of 78, 115, 71, and 86 u mol/L, respectively) in a dose dependent manner. The similarity of IC50 values indicated that cyclooxygenase was the primary target, suppressing the generation of TXA₂ which reduces aggregation of platelet and exocytosis of ATP via binding to TXA₂ receptors. (J. Gleitz et al., Planta Med., 63(1):27-30 (1997)).

### SUMMARY

The invention is based in part on the unexpected discovery that six kavalactones, dihydrokawain, dihydromethysticin, kawain, yangonin, desmethoxyyangonin, and methylsticin (structures shown below), combinations of them, and compositions including one or more of them, exhibit osteoclast formation inhibitory activity. Thus, the invention also relates to methods of using these compounds and compositions for treating or preventing disease or disease symptoms.

The osteoclast is the unique multinucleated giant cell that is responsible for bone degradation in both physiological and pathological circumstances. It has been reported that the interaction of recently discovered proteins RANKL (Receptor Activator of NF-kB ligand), osteprotegrin (OPG) and RANK, the cognate receptor for RANKL, along with M-CSF are crucial to the formation of osteoclast. (T. Suda et al., Endocr. Rev. 13, 66-80 (1992)). The invention is based on the result of RANKL-M-CSF osteoclast formation inhibition assay shown here. An interesting and unexpected finding is that the inhibitory activity observed is not related to known anti-inflammatory activity that kava components show, including cyclooxygenase inhibitory activity. This point was confirmed in simultaneous comparison of kavalactones with various anti-inflammatory agents, including even stronger cyclooxygenase inhibitors, such as aspirin and celecoxib. None of the anti-inflammatory agents examined demonstrated any osteoclast formation inhibitory activity below 25 uM under the test conditions. This demonstrates that the kavalactone's osteoclast formation inhibitory activity is quite novel and a unique finding.

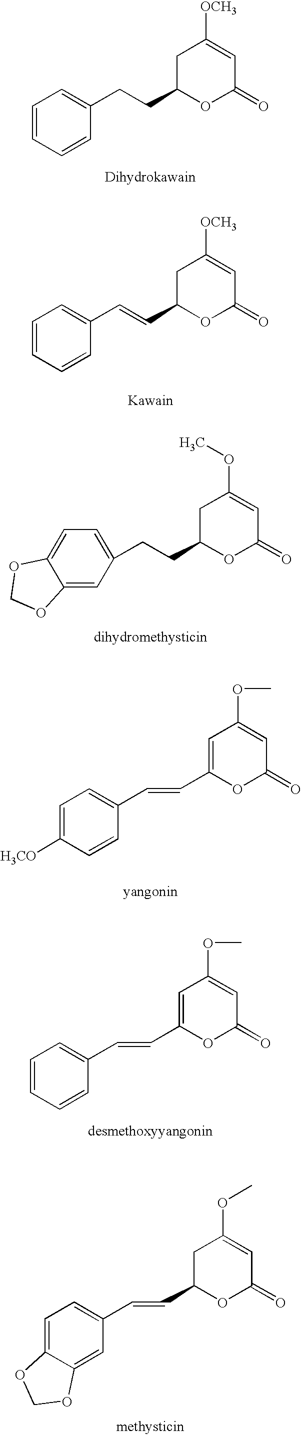

In one aspect, the invention relates to a method of treating or preventing the excessive resorption of bone in a subject comprising the administration of an effective amount of active kavalactone selected from any of dihydrokawain, dihydromethysticin, kawain, methysticin, yangonin, desmethoxyyangonin, or any combination thereof (e.g., any 2, 3, 4, 5, or all 6 of them). In the methods herein, the excessive resorption of bone is associated with rheumatoid arthritis or periodontal disease, or is associated with osteoporosis or periodontal disease.

As such, the compounds, compositions and methods of this invention are useful in treating any osteoclast-mediated disease or disease symptoms, by (or including) administration of a compound or composition herein (e.g., an effective amount of active kavalactone selected from any of dihydrokawain, dihydromethysticin, kawain, methysticin, yangonin, desmethoxyyangonin, or any combination thereof (e.g., any 2, 3, 4, 5, or all 6 of them)). In one aspect, the invention relates to a method for treating or preventing any osteoclastic bone resorption disease or disease symptom by (or including) administering an effective amount of a compound or composition as delineated herein. In another aspect the invention relates to a method for treating or preventing any disease or disease symptom. (e.g., rheumatoid arthritis, osteoporosis, osteoarthritis, periodontal diseases, Crohn's disease, Paget's disease, hyperparathyroidism, hypercalcemia, bone disease, or bone cancer) in a subject by (or including) administering an effective amount of a kavalactone compound, or composition thereof, as delineated herein. Osteoclastic bone resorption, is a key event in the pathophysiology of osteopenic diseases. Osteoclastic bone resorption involves a number of sequential events, including differentiation and activation of osteoclasts. Osteoclast-mediated disease or disease symptoms are those wherein osteoclasts are involved (e.g., proliferation, overproduction, activation) in the presentation of the disease or disease symptoms, including those delineated herein. Osteoclast-overproduction disease or disease symptoms are those wherein the overproduction of osteoclasts is involved in the disease or disease symptoms, including those delineated herein. The methods (and compounds and compositions delineated herein) are useful in treating or preventing disease or disease symptoms where mediation (e.g., inhibition, antiproliferation, control of overproduction) of osteoclasts, or their formation or activity, can result in amelioration of the disease or disease symptom.

In one aspect, the method is any method delineated herein involving administering an effective amount of active kavalactone selected from any of dihydrokawain, dihydromethysticin, kawain, methysticin, yangonin, desmethoxyyangonin, or any combination thereof (e.g., any 2, 3, 4, 5, or all 6 of them). In another aspect, the method is any delineated herein wherein the active kavalactone is a combination of dihydromethysticin, dihydrokawain, and kawain. In another aspect, the method is any delineated herein wherein the active kavalactone is yangonin, dihydrokawain, methysticin, or combination thereof.

This invention provides a medicinal oral drug because of non-toxic features of natural kavalactones. The dosage of the kavalactones according to the invention can depend on various factors such as the mode of application, species, age and/or individual conditions. Single doses can contain, for example from about 0.2 mg to 30 mg, alternatively from about 0.5 mg to 20 mg, per kilogram of body weight. Various oral dosage forms can be used, including such solid forms as tablets, gelcaps, capsules, caplets, granules, lozenges and bulk powders and liquid forms such as emulsions, solutions and suspensions. The compounds of the present invention can be administered alone or can be combined with various pharmaceutically acceptable carriers and excipients known to those skilled in the art, including but not limited to diluents, suspending agents, solubilizers, binders, retardants, disintegrants, dispersing agents, preservatives, coloring agents, lubricants and the like. When the compounds of the present invention are incorporated into oral tablets, such tablets can be compressed, tablet triturates, enteric-coated, sugar-coated, film-coated, multiply compressed, or multiply layered.

Liquid oral dosage forms include aqueous and nonaqueous solutions, emulsions, suspensions, and solutions and/or suspensions reconstituted from non-effervescent granules, containing suitable solvents, preservatives, emulsifying agents, suspending agents, diluents, sweeteners, coloring agents, or flavorings.

In one aspect, the invention relates to a medicinal ointment including 1% to 40% (e.g., 1.5% to 30%, 2% to 25%) by weight an active kavalactone selected from the group consisting of dihydrokawain, dihydromethysticin, kawain, methysticin, yangonin, desmethoxyyangonin, and a combination thereof, and a medicinally acceptable carrier. The ten “active kavalactone” herein refers only to dihydrokawain, dihydromethysticin, kawain, methysticin, yangonin, desmethoxyyangonin, or a combination of them.

In another aspect, the invention is a patch (See, for example, U.S. Pat. No. 5,186,938) including an active kavalactone-containing material layer. More specifically, the material layer, e.g., a pad or a pressure-sensitive adhesive, serves as a substrate for receiving 1% to 40% (e.g., 1.5% to 30%, 2% to 25%) by weight, an active kavalactone selected from the group consisting of dihydrokawain, dihydromethysticin, kawain, methysticin, yangonin, desmethoxyyangonin, and a combination thereof. A patch optionally having a protective layer intimately adhered to one side of the material layer, which is resistant to passage of the active kavalactone is within the invention. Also within the invention is a patch where the material layer includes a pressure-sensitive adhesive.

The invention also relates to a method for treating (e.g., curing, preventing, or ameliorating) an osteoclast overproduction-related disorder, including administering to a subject (e.g., mammal, human, dog, cat, horse) in need thereof an effective amount of an active kavalactone selected from the group consisting of dihydrokawain, dihydromethysticin, kawain, methysticin, yangonin, desmethoxyyangonin, and a combination thereof. The method of treating has an effect on the disease itself or on the symptoms. The disorders that can be treated by the method include, for example, rheumatoid arthritis, osteoporosis, osteoarthritis, Crohn's disease, Paget's disease, hypercalcemia (and related disorders), bone disease (e.g., bone cancer, see also G. A. Rodan et al., Science, 289:1508 (2000) for other examples of bone disease that the invention can be useful in treating), and periodontitis.

Another aspect of the invention relates to a packaged product including a container, a composition containing an active kavalactone disposed in the container, the kavalactone being selected from the group consisting of dihydrokawain, dihydromethysticin, kawain, methysticin, yangonin, desmethoxyyangonin, and a combination thereof, and a label (e.g., sticker, product insert) with the container and having instructions for application of the active kavalactone for treating an osteoclast overproduction-related disorder.

Also within the invention is a composition herein for use in treating disease (e.g., osteoclast-mediated diseases or disease symptoms (such as osteoarthritis), or other diseases (such as hypercalcemia or hypercalcemia-related disorders), or osteoclast overproduction-related disorders), and use of such a composition for the manufacture of a medicament for the treatment of the aforementioned diseases or disease symptoms.

The details of one or more aspects of the invention are set forth in the accompanying figure and the description below. Other features, objects, and advantages of the invention will be apparent from the description and from the claims.

### DESCRIPTION OF THE FIGURE

FIG. 1 shows the high performance liquid chromatography (HPLC) chromatograms [absorbance v. retention time (minutes)] for the mixture, and individually for each of dihydrokawain, dihydromethysticin, kawain, yangonin, desmethoxyyangonin, and methylsticin, respectively.

### DETAILED DESCRIPTION

This invention is based in part on the unexpected discovery that specific kavalactones inhibit osteoclast formation, whose overproduction is implicated in a number of diseases and disease symptoms. The osteoclast formation inhibitory activity of six major kavalactones (e.g., desmethoxyyangonin, dihydrokawain, dihydromethysticin, kawain, methysticin, and yangonin) was measured using a well-established cellular assay for determination of osteoclast formation inhibition. Among them, yangonin, dihydrokawain, and methysticin were found to have relatively higher osteoclast formation inhibitory activity relative to the other kavalactones. These results are shown in Table 1 (below). Thus, compositions containing one of the six active kavalactones or a combination thereof, are useful for treating disease or disease symptoms related to osteoclast overproduction.

This invention is also based in part on another unexpected discovery: the six active kavalactones can be administered effectively in a transdermal fashion (e.g., as a medicinal ointment). Upon homogeneous formulation in an inert carrier, the active kavalactones can be effectively administered in the absence of permeation enhancers (e.g., Dimethyl sulfoxide, 1-dodecyoazacycloheptan-2-one, sodium guaiazulene-3-sulfonate) Compositions of the invention can be administered as an ointment thus avoiding bioavailability problems associated with oral administration (e.g., first pass effects, short half-life in blood, degradation, cytochrome P450 metabolism, gut metabolism, liver or kidney metabolism, or absorption). Such administration techniques allow for systemic or local administration of the dihydrokawain, dihydromethysticin, kawain, methysticin, yangonin, desmethoxyyangonin, or a combination thereof. A medicinal ointment of the invention allows for one or more active kavalactones to reach subcutaneous levels, and provides an effect beyond that of a cosmetic or dermapharmaceutical, which affects activities at skin level (e.g., skin cell respiration, regeneration, and hydration).

An ointment composition of the invention can be formulated with one or more of the active kavalactones suspended or dissolved in a carrier, such as mineral oil, liquid petroleum, white petroleum, propylene glycol, polyoxyethylene polyoxypropylene compound, emulsifying wax, water, sorbitan monostearate, polysorbate 60, cetyl esters wax, cetyl alcohol, 2-octyldodecanol, and stearyl alcohol. An acceptable carrier can include water, a solvent, an emollient, a surfactant, a preservative, or a combination thereof. Water, when present, can be in an amount of 5 to 80% by weight. Other than water, the acceptable carrier can also contain a relatively volatile solvent such as a monohydric C1-C3 alkanol (e.g., methyl alcohol or ethyl alcohol) in an amount of 1 to 70% by weight, and an emollient such as those in the form of silicone oils and synthetic esters in an amount of 0.1 to 30% by weight. Other solvents that are acceptable carriers include any suitable for administration of dihydrokawain, dihydromethysticin, and kawain, for example, dimethyl sulfoxide, C1-C20 alcohols, glycols, and ethers. Anionic, nonionic, or cationic surfactants can also be included in the biological acceptable carrier. The concentration of total surfactants can be from 0.1 to 40% by weight. Examples of anionic surfactants include soap, alkyl ether sulfate and sulfonate, alkyl sulfate and sulfonate, alkylbenzene sulfonate, alkyl and dialkyl sulfosuccinate, C8-C20 acyl isethionate, acyl glutamate, C8-C20 alkyl ether phosphate, and a combination thereof. Examples of nonionic surfactants include C10-C20 fatty alcohol or acid hydrophobe condensed with from 2 to 100 moles of ethylene oxide or propylene oxide per mole of hydrophobe; C2-C10 alkyl phenol condensed with from 2 to 20 moles of alkylene oxide; mono and di-fatty acid ester of ethylene glycol; fatty acid monoglyceride; sorbitan, mono- and di- C8-C20 fatty acid; block co-polymer (ethylene oxide/propylene oxide); polyoxyethylene sorbitan, and a combination thereof Preservatives can also be included in the biological acceptable carrier to prevent growth of potentially harmful microorganisms, and can be employed in an amount of 0.01 to 2% by weight. Examples of preservatives include alkyl ester of para-hydroxybenzoic acid, hydantoin derivativc, propionate salt, and a variety of quaternary ammonium compounds. Each preservative should be selected based on its compatibility with other ingredients in the composition. An ointment of this invention can be applied to any particular surface area of the body (including gums).

Also within the scope of the invention is a method for treating an osteoclast overproduction-related disorder, including administering to a subject (e.g., mammal, human, dog, cat, horse) in need thereof an effective amount of an active kavalactone selected from the group consisting of dihydrokawain, dihydromethysticin, kawain, methysticin, yangonin, desmethoxyyangonin, and a combination thereof. The effective amount of active kavalactone is between 0.01 and 100 mg/kg body weight per day, alternatively between 0.5 and 75 mg/kg body weight per day of dihydrokawain, dihydromethysticin, kawain, methysticin, yangonin, desmethoxyyangonin, or a combination thereof. The effective amount is useful in a monotherapy or in combination therapy for the treatment of osteoclast overproduction-related disease or disease symptoms. As the skilled artisan will appreciate, lower or higher doses than those recited above may be required. Effective amounts and treatment regimens for any particular subject (e.g., human, dog, cat) will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician or veterinarian.

To practice the method of the present invention, an active kavalactone-containing composition can be administered can be administered orally, parenterally, by inhalation spray, topically, rectally, nasally, buccally, vaginally or via an implanted reservoir. The term “parenteral” as used herein includes subcutaneous, intracutaneous, intravenous, intramuscular, intraarticular, intraarterial, intrasynovial, intrastemal, intrathecal, intralesional and intracranial injection or infusion techniques.

A sterile injectable preparation, for example, a sterile injectable aqueous or oleaginous suspension, can be formulated according to techniques known in the art using suitable dispersing or wetting agents (such as, for example, Tween 80) and suspending agents. The sterile injectable preparation can also be a sterile injectable solution or suspension in a non-toxic parenterally acceptable diluent or solvent, for example, as a solution in 1,3-butanediol. Among the acceptable vehicles and solvents that can be employed are mannitol, water, Ringer's solution and isotonic sodium chloride solution. In addition, sterile, fixed oils are conventionally employed as a solvent or suspending medium (e.g., synthetic mono- or diglycerides). Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions can also contain a long-chain alcohol diluent or dispersant, or carboxymethyl cellulose or similar dispersing agents. Other commonly used surfactants such as Tweens or Spans or other similar emulsifying agents or bioavailability enhancers which are commonly used in the manufacture of pharmaceutically acceptable solid, liquid, or other dosage forms can also be used for the purposes of formulation.

A preparation for oral administration can be any orally acceptable dosage form including, but not limited to, capsules, tablets, emulsions and aqueous suspensions, dispersions and solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions or emulsions are administered orally, the active ingredient can be suspended or dissolved in an oily phase combined with emulsifying or suspending agents. If desired, certain sweetening, flavoring, or coloring agents can be added. A nasal aerosol or inhalation composition can be prepared according to techniques well-known in the art of pharmaceutical formulation and can be prepared as solutions in saline, employing benzyl alcohol or other suitable preservatives, absorption promoters to enhance bioavailability, fluorocarbons, and/or other solubilizing or dispersing agents known in the art. An active kavalactone-containing composition can also be administered in the form of suppositories for rectal administration. These compositions can be prepared by mixing dihydrokawain, dihydromethysticin, kawain, methysticin, yangonin, desmethoxyyangonin, or a combination thereof with a suitable non-irritating excipient which is solid at room temperature but liquid at the rectal temperature and therefore will melt in the rectum to release the active components. Such materials include, but are not limited to, cocoa butter, beeswax and polyethylene glycols.

An active kavalactone-containing composition can be administered using an implantable device. Implantable devices and related technology are known in the art and are useful as delivery systems where a continuous, or timed-release delivery of pure kavalactone compounds or compositions delineated herein is desired. Additionally, the implantable device delivery system is useful for targeting specific points of pure kavalactone compound or composition delivery (e.g., localized sites, organs). Negrin et al., Biomaterials, 22(6):563 (2001). Timed-release technology involving alternate delivery methods can also be used in this invention. For example, timed-release formulations based on polymer technologies, sustained-release techniques and encapsulation techniques (e.g., polymeric, liposomal) can also be used for delivery of the pure kavalactone compounds and compositions delineated herein. Topical-patches having pure dihydrokawain, dihydromethysticin, kawain, methysticin, yangonin, desmethoxyyangonin, or a combination thereof, or a composition thereof are also included in this invention.

Also within the invention is a patch to deliver active kavalactone. A patch includes a material layer (e.g., polymeric, cloth, gauze, bandage) and 1% to 40% by weight an active kavalactone selected from dihydrokawain, dihydromethysticin, kawain, methysticin, yangonin, desmethoxyyangonin, or a combination thereof. One side of the material layer can have a protective layer adhered to it to resist passage of active kavalactone compositions. The patch can additionally include an adhesive to hold the patch in place on a subject. An adhesive is a composition, including those of either natural or synthetic origin, that when contacted with the skin of a subject, temporarily adheres to the skin. It can be water resistant. The adhesive can be placed on the patch to hold it in contact with the skin of the subject for an extended period of time. The adhesive can be made of a tackiness, or adhesive strength, such that it holds the device in place subject to incidental contact, however, upon an affirmative act (e.g., ripping, peeling, or other intentional removal) the adhesive gives way to the external pressure placed on the device or the adhesive itself, and allows for breaking of the adhesion contact. The adhesive can be pressure sensitive, that is, it can allow for positioning of the adhesive (and the device to be adhered to the skin) against the skin by the application of pressure (e.g., pushing, rubbing,) on the adhesive or device. Also included are peelable masks that can be formulated by placing the composition as a gel or paste on a protective layer made of a film-forming polymer (e.g., polyvinyl alcohol) and an adhesive promoting polymer (e.g., hydrophobic acrylate or methacrylate polymer, such as Pemulen TR2.RTM. from the B. F. Goodrich Company). Alternatively, a hydrogel composition (See, for example, U.S. Pat. No. 5,961,479 or U.S. Pat. No. 5,306,504) including any one or more of the active kavalactones can be used.

Acceptable carriers that can be used to prcpare active kavalactone-containing compositions of this invention include, but are not limited to, ion exchangers, alumina, aluminum stearate, lecithin, self-emulsifying drug delivery systems (SEDDS) (such as d-α- tocopherol polyethyleneglycol 1000 succinate), surfactants used in pharmaceutical dosage forms (such as Tweens or other similar polymeric delivery matrices), buffer substances (such as phosphates), glycine, sorbic acid, potassium sorbate, partial glyceride mixtures of saturated vegetable fatty acids, water, salts or electrolytes (such as protamine sulfate), disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Other solubilizing agents can also be advantageously used to enhance delivery of dihydrokawain, dihydromethysticin, kawain, methysticin, yangonin, desmethoxyyangonin, or a combination thereof.

The six active kavalactones (e.g., dihydrokawain, dihydromethysticin, kawain, methysticin, yangonin, and desmethoxyyangonin) can contain one or more asymmetric centers and thus can occur as racemates and racemic mixtures, single enantiomers, individual diastereomers and diastereomeric mixtures. They can also occur in cis- or trans- or E- or Z- double bond isomeric forms. All such isomeric forms can be tested using osteoclast inhibition activity assays (e.g., in vitro, cellular) known in the art to determine their inhibitory activity, including those exemplified herein.

The invention also covers a pharmaceutical composition having a pure active kavalactone selected from dihydrokawain, dihydromethysticin, kawain, methysticin, yangonin, desmethoxyyangonin, or a combination thereof. Such a composition is useful for treating osteoclast-mediated disease or disease symptoms, or other diseases (such as hypercalcemia or hypercalcemia-related disorders, periodontal disease, Crohn's disease, Paget's disease, hyperparathyroidism, bone disease). Also within this invention is a method of treating disease or disease symptoms, (including osteoclast-mediated disease or disease symptoms) in a subject by administering to the subject a pure kavalactone-containing composition. The subject can be a human or an animal (e.g., dog, cat, horse). The term “pure” refers to a level of 90% or higher. Pure active kavalactone can be derived from natural (e.g., root extract and purification) or synthetic (e.g., synthesis from natural or synthetic materials) means, or a combination thereof.

A crude extract of the kava roots can be used as the source of active kavalactones (e.g., the six delineated herein) for the preparation of a composition of this invention. Alternatively, the active kavalactones can be further purified by column chromatography. Active kavalactones can also be obtained using other extraction methods (e.g., supercritical fluid extraction). They can also be synthesized from readily available starting materials by conventional chemical methods. See, for example, Kostermans, Reclk. Trav. Chim. Pays-Bas., 70, 79 (1951); Klohs et al., J. Org. Chem., 24, 1829 (1959); Spino et al., Tetrahedron Lett., 37, 6503 (1996), and references cited in each. The active kavalactones present in a composition can be enriched by addition of those kavalactones (from either natural or synthetic sources).

In order that the invention described herein may be more readily understood, the following examples are set forth. It should be understood that these examples are for illustrative purposes only and are not to be construed as limiting this invention in any manner. All references cited herein are expressly incorporated by reference in their entirety.

### EXAMPLE 1

A crude EtOH extract of kava-kava (100 g) containing about 40 g of kavalactones (PureWorld botanicals, NJ) was suspended into a mixture of water (300 mL) and ethyl acetate (200 mL). After removal of insoluble residues, the organic layer was separated from the aqueous layer. The aqueous layer was further extracted with ethyl acetate (200 mL×2) to produce organic extracts. All organic extracts were combined to obtain an organic solution, which was washed with a saturated NaCl solution (200 mL×2), dried over anhydrous NaSO₄, and dried. The resulting dark brown oil (45 g) was purified by column chromatography with 800 g of Kieselgel 60 (230-400 mesh ASTM, EM Science, Germany), n-hexane/ethyl acetate (2:1) being the eluting solvent. Pale yellow kavalactone fractions were collected and dried to produce a partially crystallized amorphous oil (36 g). The total content of the kavalactones in the product thus obtained was about 93% by weight. Each of the six kavalactones, dihydrokawain, dihydromethysticin, kawain, methysticin, yangonin, and desmethoxy yangonin was identified by high pressure liquid chromatography. The pale yellow kavalactone fraction (15 g) containing the six kavalactones was separated by column chromatography with 600 g of Kieselgel 60 (230-400 mesh ASTM, EM service, Germany), n-hexane/ethyl acetate (4:1) being eluting solvent to isolate six kavalactone, respectively. The amount of isolated each kavalactone is as follows:

(1) kawain: 2.5 g

(2) dihydrokawain: 4 g

(3) yangonin: 0.8 g

(4) dihydromethysticin: 3 g

(5) desmethoxyyangonin: 1 g

(6) methysticin: 1 g

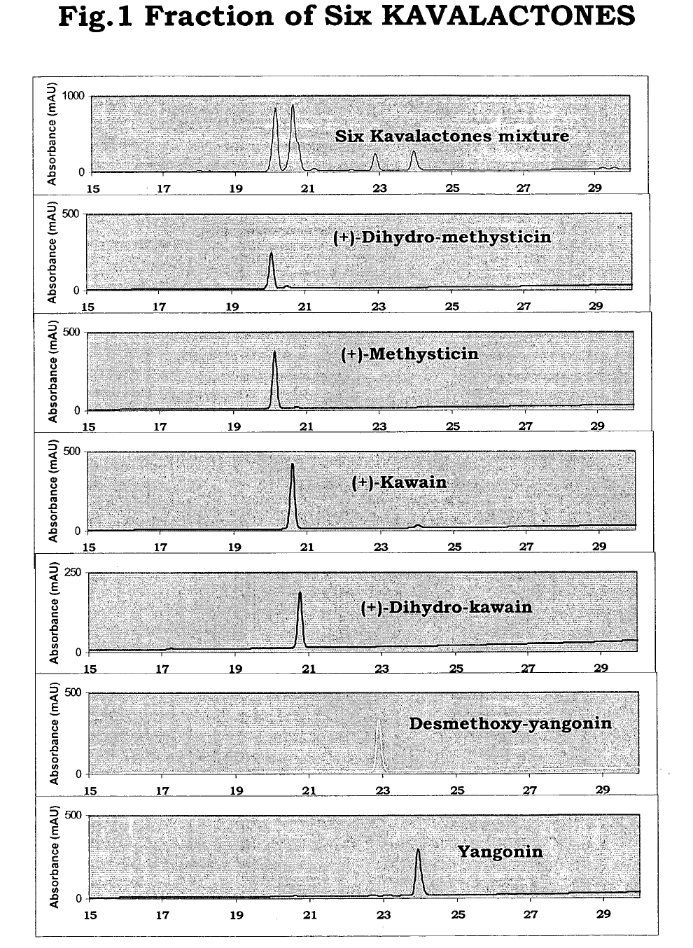

The each fraction's purity was confirmed by HPLC analysis, as seen in FIG. 1.

### EXAMPLE 2

Each of the kavalactones isolated in Example 1 was studied in an in vitro osteoclast formation assay. The protocol used (essentially that described in T. Yoneda et al., Endocrinology, 129(2):683-689 (1991) and H. Zhou et al., J Biol. Chem., 272, 14926 (2001)) is as follows:

(A) Osteoclast Formation Culture

1) Prepare assay wells (n=4) for each dilution of a test compound in a 384-plate. Each well contains 25 μl of 2× conc. of the compound in 10% FBS-media/0.5% DMSO.

2) Suspend PBMC in 10% FBS-media supplemented with 40 ng/ml of RANKL and 20 ng/ml of human M-CSF at 1.5 M cells/ml.

3) Vortex cell suspension and filtrate through mesh filter to remove cell aggregates.

4) Inoculate 25 μl of cell suspension to each well.

5) Centrifuge the assay plate at 1500 rpm (˜700 g) for 1 min.

6) Start osteoclast formation culture in a 5% CO₂-incubator.

7) After 48 h of the culture, replenish 10 μl of 10% FBS-media supplemented with 120 ng/ml of RANKL and 60 ng/ml of M-CSF. Continue the culture until multiple osteoclasts are visible in control wells (up to 4˜5 days).

(B) MTS/TRAP Assay

1) Add 6 μl of MTS reagent to each well and further incubate 1˜2 hrs.

2) Measure absorbance at 490 nm.

3) Discard MTS/culture media and fix cells in 60% acetone/0.38 M citrate buffer, pH 5.4 for 30 sec. Rinse cells with MQ once and air-dried.

4) Incubate each well in 25 μl of 5 mM p-nitrophenyl phosphate/tartrate/acetic acid solution for 2˜3 hrs (divert buffers from acid phosphate, leukocyte kit from Sigma #386-A).

5) Measure absorbance at 405 nm.

6) Discard p-nitrophenyl phosphate solution, rinse wells with MQ and stain cells with the acid phosphate, leukocyte kit.

7) Count multinucleate (>3) TRAP#-positive cell number under microscope. # Tartarate Resistant Acid Phosphatase

Under these conditions, almost complete inhibition of osteoclasts was observed with yangonin at 10 μM, methylsticin at 15 μM and dihydrokawain at 15 μM, whereas no inhibition was observed with aspirin treatment at 25 μM.

### EXAMPLE 3

Table 1 shows osteoclast formation inhibitory assay data according to the protocol described in Example 2. It is clear that popular cyclooxygenase inhibitors, aspirin and celecoxib (Entry 7 and 8) and FK 506 and cyclosporin A (Entry 9 and 10), popular immunosuppressants, do not suppress osteoclast formation under the test conditions. The validity of these experiments was demonstrated by testing two control compounds, an endogeneous osteoclast formation inhibitor, IL-4 (Bendixen et al., Proc. Natl. Acad. Sci., 98, 2443-2448 (2001) and a synthetic osteoclast formation inhibitor, tamoxifen (Shevde et al., Proc. Natl. Acad. Sci., 97, 7829-7834 (2000)). Both inhibitors (Entry 11 and 12) showed consistent activity in the experiment.

TABLE 1

| Entry | designation | IC50 | |
|---|---|---|---|
| 1 | dihydrokawain | 10 | μM |
| 2 | methysticin | 13 | μM |
| 3 | desmethoxyyangonin | 32 | μM |
| 4 | dihydromethysticin | 13 | μM |
| 5 | yangonin | 8 | μM |
| 6 | kawain | 28 | μM |
| 7 | aspirin | >100 | μM |
| 8 | celecoxib | >100 | μM |
| 9 | FK506 | >25 | μg/ml |
| 10 | cyclosporin A | >25 | μg/ml |
| 11 | IL-4 | <1 | ng/ml |
| 12 | tamoxifen | <100 | nM |

The details of one or more embodiments of the invention are set forth in the accompanying drawings and the description below. Other features, objects, and advantages of the invention will be apparent from the description and drawings, and from the claims.

What is claimed is:

1. A method of treating or preventing the excessive resorption of bone in a subject comprising the administration of an effective amount of active kavalactone selected from the group consisting of dihydrokawain, dihydromethysticin, kawain, methysticin, yangonin, desmethoxyyangonin and a combination thereof.

2. The method of claim 1, wherein the excessive resorption of bone is associated with rheumatoid arthritis or periodontal disease.

3. The method of claim 1, wherein the excessive resorption of bone is associated with osteoporosis or periodontal disease.

4. The method of claim 1, wherein the active kavalactone is a combination of dihydromethysticin, dihydrokawain, and kawain.

5. A method of treating or preventing disease or disease symptoms in a subject comprising the administration of an effective amount of active kavalactone selected from the group consisting of dihydrokawain, dihydromethysticin, kawain, methysticin, yangonin, desmethoxyyangonin and a combination thereof.

6. The method of claim 5, wherein the disease or disease symptoms are rheumatoid arthritis, osteoporosis, osteoarthritis, periodontal disease, Crohn's diseases, Paget's disease, hyperparathyroidism, or bone disease.

7. The method of claim 5, wherein the disease or disease symptoms are osteoclast-mediated disease or disease symptoms.

8. The method of claim 5, wherein the disease or disease symptoms are osteoclast-overproduction disease or disease symptoms.

9. A method of inhibiting osteoclast activity in a subject comprising the administration of an effective amount of active kavalactone selected from the group consisting of dihydrokawain, dihydromethysticin, kawain, methysticin, yangonin, desmethoxyyangonin and a combination thereof.

10. The method of claim 1, wherein the subject is a mammal.

11. The method of claim 1, wherein the subject is a human.

12. The method of claim 5, wherein the subject is a mammal.

13. The method of claim 5, wherein the subject is a human.

14. The method of claim 1, wherein the administration is oral.

15. The method of claim 5, wherein the administration is oral.

16. The method of claim 1, wherein the active kavalactone is yangonin, dihydrokawain, methysticin, or combination thereof.

17. The method of claim 5, wherein the active kavalactone is yangonin, dihydrokawain, methysticin, or combination thereof.
